(19) United States
(12) Patent Application Publication
(10) Pub. No.: US 2024/0238231 A1
(43) Pub. Date: Jul. 18, 2024

(54) EDTA AND EGTA FOR USE IN PRESERVING THE INTEGRITY OF THERAPEUTIC COMPOUNDS

(71) Applicant: Axcess (UK) Ltd, Liverpool (GB)

(72) Inventors: Roger R.C. New, London (GB); Glen Travers, London (GB)

(21) Appl. No.: 18/273,754

(22) PCT Filed: Jan. 21, 2022
(86) PCT No.: PCT/IB2022/050501

§ 371 Date: Jul. 21, 2023

(30) Foreign Application Priority Data
Jan. 22, 2021 (AU) 2021900145

(51) Int. Cl. A61K 31/198; A61K 9/00; A61K 31/235; A61K 31/575; A61K 33/06; A61K 45/06

(52) U.S. Cl. CPC A61K 31/198; A61K 9/0056; A61K 31/235; A61K 31/575; A61K 33/06; A61K 45/06

(57) ABSTRACT

The present invention relates to methods of preserving the integrity of peptides in the gut. In particular, it concerns the use of certain compounds as inhibitors of gut proteases.

### FIELD OF THE INVENTION

This Invention relates to methods of preserving the integrity of therapeutic compounds in the gut. In particular, it concerns the use of certain compounds as inhibitors of gut proteases.

### BACKGROUND

The following discussion is intended to facilitate an understanding of the present invention only. The discussion is not an acknowledgement or admission that any of the material referred to is or was part of the common general knowledge as at the priority date of the application.

Peptides, and in particular polypeptides such as proteins, are increasingly becoming recognised as desirable agents for the treatment of diseases manifesting in the gut (gastrointestinal tract). Protein therapeutics are often based on natural products with a long history of medicinal use, which have a higher safety profile than small molecules that have been newly synthesised and whose effects on the body are largely unknown. Also, proteins can exhibit a high degree of specificity and selectivity, and at the same time can be designed to take advantage of their large size to display multi-functionality, enabling them to interact concurrently with two or more different targets.

Proteins with antioxidant activity, such as superoxide dismutase, are examples of such therapeutic applications. Other examples are monoclonal antibodies, which can act as anti-infectives by binding to sites on infectious organisms invading the gut. Alternatively, such antibodies can bind to receptor sites on intestinal cells and interfere with adhesion processes and the colonisation of infectious organisms. In addition, these antibodies can interact with cells of the immune system to stimulate their activity in combating infectious diseases. Other types of therapeutic peptides include peptide hormones, such as appetite suppressing agents.

One important drawback to the use of peptides in the intestine is their extreme sensitivity to gut proteases. These proteases can be found both in the stomach (e.g. pepsin), and in the upper intestine, and have evolved to enable the digestive tract to break down peptides ingested as food, by proteolysis, into amino acids which can be taken up as nutrients by receptor-mediated mechanisms. Included in the list of gut proteases are serine proteases.

If the intended site of action of a therapeutic or prophylactic peptide is the small intestine, then the peptide can be protected from breakdown in the stomach by placing it inside an enteric-coated capsule, tablet or other device which resists dissolution at the low pH found in the stomach, but disintegrates at higher pH to release the peptide into the small intestine, e.g. the duodenum, jejunum or ileum. However, the action of the proteases found in the small intestine (in particular the serine protease trypsin) is such that they can rapidly break down and destroy peptides once they have been released from such a device. This clearly limits the efficacy of orally administered therapeutic peptides, and a means of preventing their degradation by trypsin would markedly enhance their performance.

Although there are many agents acting as protease inhibitors that are known to those skilled in the art, few, if any, are appropriate for this particular application. Most known inhibitors, e.g. antipain, leupeptin, are used for research purposes only, and are not acceptable for human administration. Some inhibitors, e.g. diisopropyl fluorophosphate or phenylmethyl sulphonyl fluoride have a high degree of potency, but display a very broad specificity, so there is a risk of their exerting their action in undesirable parts of the body, in addition to the gut. On the other hand, other inhibitors, such as the new class of protease inhibitors employed in the treatment of HIV, are so selective in the nature of the proteases they inhibit that they have no effect on serine proteases in the gut.

Two serine protease inhibitors that have been administered to humans are aprotinin and soybean trypsin inhibitor. However, these are relatively expensive to synthesise, and would have to be included in a medicament at such high levels that the cost of the final product would prohibit the manufacture of a medicament for routine daily use.

The present invention seeks to provide an alternate agent as a protease inhibitor, which at least ameliorates one or more the problems associated with prior art protease inhibitors.

### SUMMARY OF THE INVENTION

The inventors have discovered a principal of general application that was hitherto unknown. They have identified that certain compounds, whose interaction with gut proteases has never previously been recognised, surprisingly, inhibit gut proteases. This discovery proffers a hitherto unknown application of the use of these compounds as protective agents for therapeutic compounds such as for example peptides from proteolysis (i.e. degradation by gut proteases, such as trypsin, chymotrypsin, dipeptidyl peptidase 4 (DPP4) and elastase).

The inventors have discovered that EDTA or EGTA either in the presence or absence of calcium or magnesium, demonstrate marked activity in inhibiting trypsin, a well-known serine protease found in mammalian intestines. The inventors also discovered that EDTA or EGTA alone or in the presence of either calcium or magnesium also demonstrate a marked activity in inhibiting other serine proteases, such as chymotrypsin and elastase. This inhibitory activity is also expected to inhibit the serine protease, DPP4. This discovery provides new methodologies for inhibition of the activity of serine proteases.

The activity of the agents described herein is not concerned with the chelating ability of these molecules, since (i) the function of the serine proteases, such as trypsin, chymotrypsin, elastase and DPP4 is not dependent on the presence of metal ions that can be removed by chelation, (ii) agents with similar chelating activity to EDTA or EGTA display no inhibitory effect, and (iii) EDTA and EGTA works both in the presence of absence of certain metal ions.

It is proposed that the invention described herein may be used in or with pharmaceutical formulations where inhibition of trypsin-like proteases is required for effective treatment of a disease, for example in diseases of the clotting cascade, for alleviation of pathogenic sequelae of snake bite or spider envenomation, for reducing local effects of inflammation, for combatting the effects of alpha-1 anti-trypsin deficiency (e.g. in the lung), for avoiding localized side-effects of increased trypsin concentrations resulting from pancreatic disease, and for protection from proteases of therapeutic compounds, including therapeutic proteins introduced into the intestine.

In a first aspect, the invention provides a composition comprising EDTA and or EGTA for the inhibition of serine protease activity. In a preferred embodiment of this aspect of the invention, the composition inhibits serine protease activity to preserve the integrity of one or more therapeutic compounds in the gut.

In a second aspect, the invention provides a composition comprising EDTA and or EGTA and a metal ion, wherein the molar ratio of the metal ion to EDTA or EGTA is at least 1:1, and preferably 1:2, 1:1, 1:0.5, 1:0.25. In another preferred embodiment, the metal ion is calcium or magnesium.

In an alternate preferred embodiment, this aspect of the invention provides a composition comprising EDTA or EGTA, or a pharmaceutically acceptable salt thereof (preferably a calcium salt) that produces in the intestine of a patient, a concentration of EDTA or EGTA, or a pharmaceutically acceptable salt thereof (preferably a calcium salt) of between 0.1 to 10 mg/ml. Preferably, in a concentration of between 0.15 to 10 mg/ml, more preferably in a concentration of between 0.2 to 5 mg/ml or 0.2 to 1 mg/ml or even more preferably in a concentration of between 0.2 to 0.3 mg/ml. Thus, for example, in a capsule containing approximately 100 mg of solid powder excipients which dissolve readily in 1 ml or less of liquid situated in the lumen of the gut, there may be provided from 0.2 to 10 mg of EDTA or EGTA.

In another preferred embodiment, this aspect of the invention provides a composition comprising: chenodeoxycholate and/or propyl gallate, EDTA or EGTA, or a pharmaceutically acceptable salt thereof (preferably a calcium salt) that produces in the intestine of a patient, a concentration of EDTA or EGTA, or a pharmaceutically acceptable salt thereof (preferably a calcium salt) of between 0.1 to 10 mg/ml. Preferably, in a concentration of between 0.15 to 10 mg/ml, more preferably in a concentration of between 0.2 to 5 mg/ml or 0.2 to 1 mg/ml or even more preferably in a concentration of between 0.2 to 0.3 mg/ml. Thus, for example, in a capsule containing approximately 100 mg of solid powder excipients which dissolve readily in 1 ml or less of liquid situated in the lumen of the gut, there may be provided from 0.2 to 10 mg of EDTA or EGTA.

In a third aspect, the invention provides a pharmaceutical or therapeutic composition comprising EDTA and or EGTA and a metal ion, wherein the molar ratio of metal ion to EDTA or EGTA is at least 1:1. Preferably, the molar ratio of metal ion to EDTA or EGTA ranges from 1:2, to 1:1, 1:0.5, 1:0.25. In another preferred embodiment, the metal ion is calcium or magnesium.

In an alternate embodiment, the invention provides a pharmaceutical or therapeutic composition comprising EDTA and or EGTA and a metal ion, wherein the molar ratio of metal ion to EDTA or EGTA is at least 1:1, and preferably 1:2, to 1:1, 1:0.5, 1:0.25, and a therapeutically effective amount of chenodeoxycholate and/or propyl gallate. In highly preferred embodiment, the metal ion is calcium or magnesium.

In a fourth aspect, the invention provides for the use of a composition comprising EDTA and or EGTA and calcium, wherein the molar ratio of calcium to EDTA or EGTA is at least 1:1, and preferably 1:2, 1:1, 1:0.5, 1:0.25, in the manufacture of a medicament for the treatment of an ailment requiring the inhibition of trypsin or trypsin-like proteases. For example, the medicament is used for treatment of diseases of the clotting cascade, for alleviation of pathogenic sequelae of snake bite or spider envenomation, for reducing local effects of inflammation, for combatting the effects of alpha-1 anti-trypsin deficiency (e.g. in the lung), for avoiding localized side-effects of increased trypsin concentrations resulting from pancreatic disease, and for protection from proteases of therapeutic compounds, such as therapeutic proteins introduced into the intestine.

In an alternate preferred aspect, the invention provides for the use of a composition comprising EDTA and or EGTA and magnesium, wherein the molar ratio of magnesium to EDTA or EGTA is at least 1:1 in the manufacture of a medicament for the treatment of an ailment requiring the inhibition of trypsin or trypsin-like proteases. For example, the medicament is used for treatment of diseases of the clotting cascade, for alleviation of pathogenic sequelae of snake bite or spider envenomation, for reducing local effects of inflammation, for combatting the effects of alpha-1 anti-trypsin deficiency (e.g. in the lung), for avoiding localized side-effects of increased trypsin concentrations resulting from pancreatic disease, and for protection from proteases of therapeutic compounds, such as therapeutic proteins introduced into the intestine.

In an alternate preferred aspect, the invention provides for the use of a composition comprising a therapeutically effective amount of chenodeoxycholate and/or propyl gallate, EDTA and or EGTA, and calcium or magnesium, wherein the molar ratio of calcium or magnesium to EDTA or EGTA is at least 1:1, and preferably 1:2, 1:1, 1:0.5, 1:0.25, in the manufacture of a medicament for the treatment of an ailment requiring the inhibition of trypsin or trypsin-like proteases. For example, the medicament is used for treatment of diseases of the clotting cascade, for alleviation of pathogenic sequelae of snake bite or spider envenomation, for reducing local effects of inflammation, for combatting the effects of alpha-1 anti-trypsin deficiency (e.g. in the lung), for avoiding localized side-effects of increased trypsin concentrations resulting from pancreatic disease, and for protection from proteases of therapeutic compounds, such as therapeutic proteins introduced into the intestine.

In an alternate preferred form, this aspect of the invention provides for the use of: (i) a therapeutic compound; and (ii) EDTA or EGTA, or calcium salts thereof, in the manufacture of a medicament, wherein said EDTA or EGTA are present in the composition at a level giving rise to a concentration of 0.15 to 10 mg/ml and the composition is formulated for the treatment of an ailment requiring the inhibition of a serine protease, including trypsin, chymotrypsin, elastase and DPP4.

In a fifth aspect, the invention provides for the use of a composition comprising EDTA and or EGTA and calcium, wherein the molar ratio of calcium to EDTA or EGTA is at least 1:1, and preferably 1:2, 1:1, 1:0.5, 1:0.25, in the manufacture of a medicament for the treatment of an ailment, wherein the medicament includes a therapeutic compound and the composition is present in an amount that protects said a therapeutic compound from proteases in the intestine.

In an alternate preferred embodiment, this aspect of the invention provides for the use of: (i) a therapeutic compound; and (ii) EDTA or EGTA, or calcium salts thereof, in the manufacture of a medicament, wherein said EDTA or EGTA are present in the composition at a level giving rise to a EDTA or EGTA concentration of 0.15 to 10 mg/ml and the composition is formulated for delivery of the compound in the intestinal tract. Preferably, the EDTA or EGTA concentration is between 0.15 to 10 mg/ml, more preferably between 0.2 to 5 mg/ml or 0.2 to 1 mg/ml or even more preferably between 0.2 to 0.3 mg/ml.

In an alternate preferred embodiment, this aspect of the invention provides for the use of: (i) a therapeutic compound; (ii) a therapeutically effective amount of chenodeoxycholate and/or propyl gallate, and (iii) EDTA or EGTA, or calcium salts thereof, in the manufacture of a medicament, wherein said EDTA or EGTA are present in the composition at a level giving rise to a EDTA or EGTA concentration of 0.15 to 10 mg/ml and the composition is formulated for delivery of the compound in the intestinal tract. Preferably, the EDTA or EGTA concentration is between 0.15 to 10 mg/ml, more preferably between 0.2 to 5 mg/ml or 0.2 to 1 mg/ml or even more preferably between 0.2 to 0.3 mg/ml.

In a sixth aspect, the invention provides a method for treating a subject suffering from an ailment, wherein the subject's treatment is affected by the inhibition of a serine protease, including trypsin, chymotrypsin, elastase, or DPP4, said method comprising the step of: administering to a subject a composition comprising EDTA and or EGTA and calcium or magnesium, wherein the molar ratio of calcium or magnesium to EDTA or EGTA is at least 1:1, and preferably 1:2, 1:1, 1:0.5, 1:0.25. Preferably, the composition is provided in a pharmaceutically or therapeutically effective amount.

In an alternate preferred embodiment, this aspect of the invention provides a method for treating a subject suffering from an ailment, wherein the subject's treatment is effected by the inhibition of a serine protease, including trypsin, chymotrypsin, elastase or DPP4, said method comprising the step of: administering to a subject EDTA or EGTA, or calcium salts thereof, wherein said EDTA or EGTA are present in the composition at a level giving rise to a EDTA or EGTA concentration of 0.15 to 10 mg/ml.

In a seventh aspect, the invention provides a method for treating a subject suffering from an ailment, said treatment comprising administering to a subject a therapeutic compound to treat the ailment and at substantially the same time administering a therapeutically or pharmaceutically acceptable composition comprising EDTA and or EGTA and calcium or magnesium, wherein the molar ratio of calcium or magnesium to EDTA or EGTA is at least 1:1, and preferably 1:2, 1:1, 1:0.5, 1:0.2, wherein said composition is present in an amount that protects said therapeutic compound from at least a serine protease in the intestine.

In an alternate preferred embodiment, this aspect of the invention provides a method for treating a subject suffering from an ailment, said treatment comprising administering to a subject a therapeutic compound to treat the ailment and at substantially the same time administering a therapeutically or pharmaceutically acceptable composition comprising EDTA or EGTA, or calcium salts thereof, wherein said EDTA or EGTA are present in the composition at a level giving rise to a concentration of 0.15 to 10 mg/ml and the composition is formulated for delivery of the compound in the intestinal tract.

In an alternate preferred embodiment, this aspect of the invention provides a method for treating a subject suffering from an ailment, said treatment comprising administering to a subject a therapeutic compound to treat the ailment and at substantially the same time administering a therapeutically or pharmaceutically acceptable composition comprising EDTA or EGTA, or calcium salts thereof, wherein said EDTA or EGTA are present in the composition at a level giving rise to a concentration of 0.15 to 10 mg/ml, a therapeutically effective amount of chenodeoxycholate and/or propyl gallate and the composition is formulated for delivery of the compound in the intestinal tract.

These and/or other aspects of the invention will become apparent and more readily appreciated from the following description of the exemplary embodiments, taken in conjunction with the accompanying figures.

### BRIEF DESCRIPTION OF THE DRAWINGS

The disclosure will provide details in the following description of preferred embodiments with reference to the following figures wherein:

FIG. 1: Inhibition of Trypsin Activity by EDTA at Different Concentrations in the Presence of Calcium Ions.

FIG. 2: Inhibition of Trypsin Activity by EGTA at Different Concentrations in the Presence and Absence of Calcium Ions.

FIG. 3: Effect on Trypsin Activity of EGTA and Phenanthroline at Different Concentrations in the Presence of Calcium Ions.

FIG. 4: Inhibition of Chymotrypsin Activity by EDTA at Different Concentrations in the Presence of Calcium Ions.

### DETAILED EMBODIMENTS OF THE INVENTION

The inventors have discovered that EDTA or EGTA either alone or when in the presence of calcium or magnesium demonstrate marked activity in inhibiting serine proteases, including trypsin, chymotrypsin, elastase or DPP4, well-known serine proteases found in mammalian intestines. This discovery provides new methodologies for inhibition of the activity of serine proteases.

The activity of the agents described herein is not concerned with the chelating ability of these molecules, since (i) the function of the serine protease (such as trypsin) is not dependent on the presence of metal ions that can be removed by chelation, (ii) agents with similar chelating activity to EDTA or EGTA display no inhibitory effect, and (iii) EDTA and EGTA works both in the absence or presence of certain metal ions. In particular, the inventors have learnt that at the claimed molar ratio EDTA is not just removing calcium, and reducing the serine protease (such as trypsin) activity indeed the serine protease (such as trypsin) activity increases as the concentration of EDTA is increased beyond the claimed molar ratio. Further citrate, which also chelates calcium, has no effect on trypsin activity or the activity of other serine proteases. The inventors have examined this with trypsin both pre-activated with calcium, and not pre-activated. The inventors believe that in nature trypsin is in the active calcium form, as well as in other metal ion activated forms, in the gut. It is also believed that other serine proteases, such as chymotrypsin, elastase and DPP4 are also in the active calcium form or other metal ion activated form in the gut.

It is proposed that the invention described herein may be used in or with pharmaceutical formulations where inhibition of trypsin-like proteases is required for effective treatment of a disease, for example in diseases of the clotting cascade, for alleviation of pathogenic sequelae of snake bite or spider envenomation, for reducing local effects of inflammation, for combatting the effects of alpha-1 anti-trypsin deficiency (e.g. in the lung), for avoiding localized side-effects of increased trypsin concentrations resulting from pancreatic disease, and for protection from proteases of therapeutic proteins introduced into the intestine.

For convenience, the following sections generally outline the various meanings of the terms used herein. Following this discussion, general aspects regarding compositions, use of medicaments and methods of the invention are discussed, followed by specific examples demonstrating the properties of various embodiments of the invention and how they can be employed.

### Definitions

The meaning of certain terms and phrases used in the specification, examples, and appended claims, are provided below. If there is an apparent discrepancy between the usage of a term in the art and its definition provided herein, the definition provided within the specification shall prevail.

Those skilled in the art will appreciate that the invention described herein is susceptible to variations and modifications other than those specifically described. The invention includes all such variations and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness. None of the cited material or the information contained in that material should, however be understood to be common general knowledge.

Manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

Other than in the operating examples, or where otherwise indicated, all numbers expressing quantities of ingredients or reaction conditions used herein should be understood as modified in all instances by the term “about.” The term “about” when used in connection with percentages can mean±1%.

The invention described herein may include one or more range of values (e.g. size, concentration etc.). A range of values will be understood to include all values within the range, including the values defining the range, and values adjacent to the range which lead to the same or substantially the same outcome as the values immediately adjacent to that value which defines the boundary to the range. For example, a person skilled in the field will understand that a 10% variation in upper or lower limits of a range can be totally appropriate and is encompassed by the invention. More particularly, the variation in upper or lower limits of a range will be 5% or as is commonly recognised in the art, whichever is greater.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

Throughout this specification, unless the context requires otherwise, the word “comprise” or variations such as “comprises” or “comprising”, will be understood to imply the inclusion of a stated integer or group of integers but not the exclusion of any other integer or group of integers.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, e.g. in the absence of an agent, for example a decrease by at least about 20%, or at least about 30%, or at least about 40%, or at least about 50%), or at least about 60%>, or at least about 70%, or at least about 80%.

The terms “increased”, ‘increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, e.g. in in the absence of an agent, for example an increase of at least about 20%, or at least about 30%, or at least about 40%, or at least about 50%), or at least about 60%, or at least about 70%, or at least about 80%, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration. In certain embodiments, the compound is administered by parenterally administration, or other method allowing delivery to a target site.

As used herein, the terms “EDTA” or “EGTA” are used herein to generally include pharmaceutical acceptable salts thereof.

As used herein, the terms “molar ratio of EDTA:Ca”, “molar ratio of EDTA:Mg”, “molar ratio of EGTA:Ca” and “molar ratio of EGTA:“Mg” has no free divalent metal ion. Since EDTA and EGTA bind a maximum of two divalent metal ions, the ratio range includes from 1:2, 1:1, 1:0.5, 1:0.25 and so on.

As used herein, the term serine protease may be defined by the catalytic triad. The triad is located in the active site of the enzyme, where catalysis occurs, and is preserved in all families of serine protease enzymes. The triad is a coordinated structure consisting of three amino acids: His 57, Ser 195, and Asp 102. These three key amino acids each play an essential role in the cleaving ability of the proteases. The particular geometry of the triad members are highly characteristic to their specific function. Serine proteases include trypsin, chymotrypsin, elastase and dipeptidyl peptidase 4 (DPP4).

Generally, the present invention is concerned with the treatment of humans, e.g. where “gut” is mentioned it typically refers to the human gut, although in one aspect the invention concerns the treatment of non-human animals. Accordingly the subject in whom the present invention may find specific utility includes, by way of illustration: humans, mammals, companion animals and birds.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Features of the invention will now be discussed with reference to the following non-limiting description and examples.

### EMBODIMENTS

The compounds identified as exhibiting this activity are either EDTA or EGTA alone or as a combination of EDTA or EGTA with calcium or magnesium. Chelating agents, of which these are examples, are known to be able to inhibit the activity of certain metallo-enzymes by virtue of their ability to bind to metal ions, abstracting them form the metal-binding site in the enzyme's active centre, to leave an apo-enzyme which is inactive. In the case of trypsin as well as other serine proteases such as chymotrypsin, elastase and DPP4, however, these enzymes are not a metallo-protein, so inhibition of activity by a chelating agent would not be expected. Indeed, the general consensus of opinion is that chelating agents can enhance the activity of trypsin and other serine proteases, and on this basis the use of EDTA in conjunction with trypsin or other serine proteases is recommended when using this enzyme to remove cells from plastic surfaces in culture.

It has now been found that EDTA and EGTA are able to inhibit the activity of trypsin, but only in the presence of calcium ions or magnesium ions to a greater extent than EDTA or EGTA alone. However, it was also found that EDTA and EGTA are able to inhibit the activity of trypsin and chymotrypsin in the absence of calcium ions or magnesium ions, albeit to a lesser amount that when in the presence of calcium or magnesium ions. The fact that such metal ions need to be present for the increased inhibition to manifest itself suggests clearly that EGTA and EDTA are not acting as classical chelating agents, since if that were the case, then addition of metal ions would counteract the effect. It is also important to note that other commonly used chelating agents such as citric acid (in the form of the sodium salt) or ortho-phenanthroline, do not shown this inhibitory effect. The inhibition also appears to be specific to particular metal ions—calcium and magnesium showing inhibition, while zinc has no such effect, and even appears to enhance trypsin activity. Finally, it is important to note that the optimum molar ratio of EDTA to calcium or magnesium appears to be in the range of 1:1, suggesting that one possible mechanism of action could involve specific inhibition by a complex of EDTA or EGTA with the divalent metal ion in a ratio of one atom of calcium or magnesium to one of EDTA or EGTA. Preferably, for the molar ratio of EDTA:Ca, EDTA:Mg, EGTA:Ca or EGTA:Mg, it is important that there is no free metal ion, so since EDTA or EGTA binds a maximum of two calcium ions or two magnesium ions, this means that the ratio may range from 1:2, to 1:1, 1:0.5, 1:0.25 and so on. Preferably, the ratio ranges between 1:1 and 1:0.25.

### A. Compositions

Accordingly, the invention is directed to a compound for use as an inhibitor of a serine protease, selected from the group comprising trypsin, chymotrypsin, elastase and DPP4, which compound is EDTA or EGTA, or a pharmaceutically acceptable salt thereof. Preferably the compound is a calcium salt.

In a first aspect, the invention provides a composition comprising EDTA and or EGTA and calcium or magnesium, wherein the molar ratio of calcium or magnesium to EDTA or EGTA is at least 1:1.

In an embodiment, the first aspect of the invention provides a composition including EDTA and calcium or magnesium, wherein the molar ratio of EDTA to calcium or magnesium is approximately 1:2. More preferably the molar ratio of EDTA to calcium or magnesium is 1:2. More preferably the molar ratio of EDTA to calcium or magnesium ranges from 1:2, 1:1, 1:0.5, 1:0.25.

In an alternate embodiment, the first aspect of the invention provides a composition including EGTA and calcium or magnesium, wherein the molar ratio of EGTA to calcium or magnesium is approximately 1:2. More preferably the molar ratio of EGTA to calcium or magnesium is 1:2. More preferably the molar ratio of EDTA to calcium or magnesium ranges from 1:2, 1:1, 1:0.5, 1:0.25.

In a second aspect, the invention provides a pharmaceutical or therapeutic composition comprising EDTA and or EGTA and calcium or magnesium, wherein the molar ratio of calcium or magnesium to EDTA or EGTA is at least 1:1. More preferably the molar ratio of EDTA to calcium or magnesium ranges from 1:2, 1:1, 1:0.5, 1:0.25.

In an embodiment, the second aspect of the invention provides a pharmaceutical or therapeutic composition including EDTA and calcium or magnesium, wherein the molar ratio of EDTA to calcium or magnesium is approximately 1:2. More preferably the molar ratio of EDTA to calcium or magnesium is 1:2. More preferably the molar ratio of EDTA to calcium or magnesium ranges from 1:2, 1:1, 1:0.5, 1:0.25.

In an alternate embodiment, the second aspect of the invention provides a pharmaceutical or therapeutic composition including EGTA and calcium or magnesium, wherein the molar ratio of EGTA to calcium or magnesium is approximately 1:2. More preferably the molar ratio of EGTA to calcium or magnesium is 1:2. More preferably the molar ratio of EDTA to calcium or magnesium ranges from 1:2, 1:1, 1:0.5, 1:0.25.

In a preferred embodiment of the second aspect of the invention there is provided a product or pharmaceutical composition containing: (a) EDTA or EGTA or a calcium salt of either compound, and (b) a therapeutic compound, wherein (a) and (b) are prepared for simultaneous, separate or sequential delivery to a subject for the treatment of a disease or condition affecting the subject, or for preventing a disease or condition affecting the subject.

The therapeutic compound for use in accordance with the present invention may be any compound that is altered, degraded or effected by a protease that has a therapeutic effect inside a human or animal body, including inside one or more cells in the human or animal body. In this regard, and generally herein, references to a (or the) therapeutic compound for use in accordance with the invention are intended to encompass also the possibility of using more than one therapeutic compound, such as 2, 3, or more therapeutic compounds. The therapeutic compound for use in accordance with the present invention is a macromolecule. In this regard, the therapeutic compound preferably has a molecular weight of around 1000 Da or more, such as e.g. 2000 Da or more, or 3000 Da or more.

The therapeutic compound is preferably, although not essentially, a peptide, more preferably a polypeptide, and more preferably still is a protein. Examples of suitable therapeutic compounds include, without limitation, insulin; calcitonin; human serum albumin; growth hormone; growth hormone releasing factors; galanin; parathyroid hormone; peptide YY; oxyntomodulin; blood clotting proteins such as kinogen, prothombin, fibrinogen, Factor VII, Factor VIII of Factor IX; erythropoeitins and EPO mimetics; colony stimulating factors including GCSF and GMCSF; platelet-derived growth factors; epidermal growth factors; fibroblast growth factors; transforming growth factors; GLP-1, GLP-2; GLP-1 analogues and fusion proteins, GIP, glucagon; exendin; leptin; GAG; cytokines; insulin-like growth factors; bone- and cartilage-inducing factors; neurotrophic factors; interleukins including IL-I, IL-2, IL-3, IL-4, IL-5, IL-6, IL-7, IL-8, IL-9, IL-10, IL-11, IL-12; interferons including interferon gamma, interferon-1a, interferon alphas; TNF alpha; TNF beta; TGF-beta; cholera toxin A and B fragments; E. coli enterotoxin A and B fragments; secretin; enzymes including histone deacetylase, superoxide dismutase, catalase, adenosine deaminase, thymidine kinase, cytosine deaminase, proteases, lipases, carbohydrases, nucleotidases, polymerases, kinases and phosphatases; transport or binding proteins especially those which bind and/or transport a vitamin, metal ion, amino acid or lipid or lipoprotein such as cholesterol ester transfer protein, phospholipid transfer protein, HDL binding protein; connective tissue proteins such as a collagen, elastin or fibronectin; a muscle protein such as actin, myosin, dystrophin, or mini-dystrophin; a neuronal, liver, cardiac, or adipocyte protein; a cytotoxic protein; a cytochrome; a protein which is able to cause replication, growth or differentiation of cells; a signalling molecule such as an intra-cellular signalling protein or an extracellular signalling protein (e.g. hormone); trophic factors such as BDNF, CNTF5NGF, IGF, GMF, aFGF, bFGF, VEGF, NT3, T3 and HARP; apolipoproteins; antibody molecules, antibody fragments, single-domain antibodies; receptors in soluble form such as T-cell receptors and receptors for cytokines, interferons or chemokines; proteins or peptides containing antigenic epitopes and fragments; and albumin fusion proteins, derivatives, conjugates and sequence variants of any of the above. These and other proteins may be derived from human, plant, animal, bacterial or fungal sources, and extracted either from natural sources, prepared as recombinants by fermentation or chemically synthesised. In a preferred aspect, the therapeutic compound is a peptide selected from insulin, calcitonin, growth hormone, growth hormone releasing factors, galanin, parathyroid hormone, peptide YY, oxyntomodulin, erythropoeitins, colony stimulating factors, platelet-derived growth factors, epidermal growth factors, fibroblast growth factors, transforming growth factors, GLP-1, GLP-2, GIP, glucagon, exendin, leptin, neurotrophic factors, insulin-like growth factors, cartilage-inducing factors, IL-1, IL-2, IL-3, IL-4, IL-5, IL-6, IL-7, IL-8, IL-9, IL-10, IL-11, IL-12, interferon-gamma, interferon-la, interferon alphas, and conjugates of any of the above, fusion proteins including any of the above, and any of the above in combination.

Preferably the product or pharmaceutical composition is formulated for delivery to the gut of the subject.

In bodily fluids, homeostatic mechanisms act to maintain the calcium level at a concentration of 1 mM under normal physiological conditions. Consequently, for the EDTA to be active in a form where it is complexed with calcium in a ratio of 1:2 or less, it should be present at a level resulting in a concentration of 0.5 mM EDTA in solution in the gut. For the sodium EDTA salt, this will be equivalent to a level of 0.1 mg/ml or above. In the event that higher concentrations of EDTA are used, calcium levels are adjusted, so that the molar ratio continues to be 1:2 or less (EDTA:calcium). A similar adjustment applies to the use of magnesium, when and if appropriate.

In a form of the invention the product or pharmaceutical composition is present in the intestine in a concentration of between 0.1 to 10 mg/ml. Preferably, the EDTA concentration is between 0.15 to 10 mg/ml, more preferably between 0.2 to 5 mg/ml or 0.2 to 1 mg/ml or even more preferably between 0.2 to 0.3 mg/ml. For example, it may be in a concentration of between approximately 0.3 to 10 mg/ml. Alternatively the concentration can be: 0.15, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 or 10.0 mg/ml.

Although trypsin is only one of four proteases involved in the breakdown of peptides in the intestine, the ability to inhibit just this single enzyme assumes particular importance since it is responsible for activating other proteases, cleaving inactive pro-enzymes after they have passed from the pancreas into the duodenum. Indeed, trypsin even activates itself, cleaving a small stretch of peptide from the N-terminal end of the peptide bond after lysine residue 15. Consequently, inhibition of small amounts of active trypsin present in the duodenum can have a profound effect in inhibiting an amplification cascade which would normally occur as new material is secreted from the pancreas into the upper intestinal tract. In the same way, the activation of chymotrypsin, elastase and carboxypeptidase can be inhibited by a trypsin inhibitor; since chymotrypsinogen is activated by cleavage by trypsin at a site between arginine 15 and isoleucine 16 by trypsin, leading to formation of the active enzyme. Trypsin also cleaves a 12-residue peptide from the N-terminal end of proelastase, resulting in activation. Finally, pancreatic procarboxypeptidases A and B are activated by cleavage with trypsin, and inhibition of this process can prevent activation. As a result, indirectly, the inhibition of trypsin activity alone can protect proteins in the intestine which are vulnerable to attack at many different cleavage sites, and not just those which are specific to trypsin.

There is another route to activation of trypsin by cleavage of trypsinogen, namely as a result of interaction of trypsinogen with enteropeptidase, which is found on the mucosal surfaces of the intestine. Enteropeptidase is a serine protein.

Accordingly, an effective way of inhibiting the amplification cascade described above includes combining an inhibitor specific for trypsin as described herein with a general inhibitor for serine proteases.

In an embodiment, the second aspect of the invention provides a pharmaceutical or therapeutic composition including (i) EDTA or EGTA and calcium, wherein the molar ratio of EDTA to calcium is approximately 1:2 and (ii) a general inhibitor of serine protease. Preferably, the molar ratio of EDTA to calcium or magnesium ranges from 1:2, 1:1, 1:0.5, 1:0.25.

Preferably the serine protease inhibitor acts on enteropeptidase and prevents that enzyme from generating fresh trypsin, and the inhibitor specific for trypsin would act on activated enzyme already in the intestine, and prevent it from contributing to the trypsin autolytic chain reaction. Preferably, these agents would both be administered in such a way as to act in the duodenum, on a fasted stomach, before any cleavage reactions had taken place.

An example of a general inhibitor of serine proteases is chenodeoxycholate, whose activity on a range of serine proteases, including thrombin, trypsin and chymotrypsin has been reported in patent PCT/AU2009/001305. Thus, an additional feature of this invention is a composition, preferably a pharmaceutical composition of matter, comprising a combination of chenodeoxycholate with EDTA and/or EGTA alone or plus calcium or magnesium, in the preferred ratio as described above. An example of such a composition includes a capsule containing inter alia Aerosil and SSG, 70 mg of chenodeoxycholate and 33 mg of propyl gallate, wherein the capsule is coated so as to resist breakdown in the stomach, but where the coating dissolves readily at the pH of the duodenum—between 5.5 and 6.5.

In an alternate form of the invention the general inhibitor of serine proteases includes: serine protease inhibitors such as soybean trypsin inhibitor, aprotinin, Bowman-Birk inhibitor, Ecotin, natural serpins, metformin, chenodeoxycholate, deoxycholate, as well as other more specific inhibitors such as chymostatin (for chymotrypsin), elastatin, elasnin, sivelestat (for elastase), and members of the MEROPS inhibitor family, 2-MPPA, PMPA, and ZJ43 (for carboxypeptidase).

### B. Use of a Composition in the Manufacture of a Medicament

In a third aspect, the invention provides for the use of a composition comprising EDTA and or EGTA and calcium, wherein the molar ratio of calcium to EDTA or EGTA is at least 1:1, and preferably 1:1, 1:2, 1:0.05, 1:0.25, in the manufacture of a medicament for the treatment of an ailment requiring the inhibition of trypsin. Preferably, the medicament is used for treatment of a disease where inhibition of trypsin-like proteases is required for effective treatment of a disease, for example in diseases of the clotting cascade, for alleviation of pathogenic sequelae of snake bite or spider envenomation, for reducing local effects of inflammation, for combatting the effects of alpha-1 anti-trypsin deficiency (eg in the lung), for avoiding localized side-effects of increased trypsin concentrations resulting from pancreatic disease, and for protection from proteases of therapeutic proteins introduced into the intestine.

In an embodiment, the third aspect of the invention provides for the use of a composition including EDTA and calcium or magnesium, wherein the molar ratio of calcium or magnesium to EDTA is at least 1:1, and preferably 1:1, 1:2, 1:0.05, 1:0.25, in the manufacture of a medicament for the treatment of an ailment requiring the inhibition of trypsin, chymotrypsin, elastase or DPP4. Preferably, the medicament is used for treatment of a disease where inhibition of trypsin-like proteases is required for effective treatment of a disease, for example in diseases of the clotting cascade, for alleviation of pathogenic sequelae of snake bite or spider envenomation, for reducing local effects of inflammation, for combatting the effects of alpha-1 anti-trypsin deficiency (e.g. in the lung), for avoiding localized side-effects of increased trypsin concentrations resulting from pancreatic disease, and for protection from proteases of therapeutic proteins introduced into the intestine.

In an alternate embodiment, the third aspect of the invention provides for the use of a composition including EGTA and calcium or magnesium, wherein the molar ratio of calcium or magnesium to EGTA is at least 1:1, and preferably 1:1, 1:2, 1:0.05, 1:0.25, in the manufacture of a medicament for the treatment of an ailment requiring the inhibition of trypsin, chymotrypsin, elastase or DPP4. Preferably, the medicament is used for treatment of a disease where inhibition of trypsin-like proteases is required for effective treatment of a disease, for example in diseases of the clotting cascade, for alleviation of pathogenic sequelae of snake bite or spider envenomation, for reducing local effects of inflammation, for combatting the effects of alpha-1 anti-trypsin deficiency (eg in the lung), for avoiding localized side-effects of increased trypsin concentrations resulting from pancreatic disease, and for protection from proteases of therapeutic proteins introduced into the intestine.

In an alternate preferred embodiment, the invention provides for the use of: (i) a therapeutic compound; and (ii) EDTA or EGTA, or calcium salts thereof, in the manufacture of a medicament, wherein said EDTA or EGTA are present in the composition at a level giving rise to a concentration of 0.1 to 10 mg/ml and the composition is formulated for the treatment of an ailment requiring the inhibition of trypsin or a trypsin-like protease, chymotrypsin, elastase or DPP4.

In yet another alternate preferred embodiment, the invention provides for the use of: (i) a therapeutic compound; and (ii) EDTA or EGTA, or calcium salts thereof, in the manufacture of a medicament, wherein said EDTA or EGTA are present in the composition at a level giving rise to a concentration of 0.1 to 10 mg/ml and the composition is formulated for the treatment of an ailment requiring the inhibition of trypsin or a trypsin-like protease, chymotrypsin, elastase or DPP4. Preferably, the EDTA or EGTA or calcium salts thereof concentration is between 0.15 to 10 mg/ml, more preferably between 0.2 to 5 mg/ml or 0.2 to 1 mg/ml or even more preferably between 0.2 to 0.3 mg/ml. For example, it may be in a concentration of between approximately 0.3 to 10 mg/ml. Alternatively the concentration can be: 0.15, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 or 10.0 mg/ml.

In a fourth aspect, the invention provides for the use of a composition comprising EDTA and or EGTA and calcium or magnesium, wherein the molar ratio of calcium or magnesium to EDTA or EGTA is at least 1:1, and preferably 1:1, 1:2, 1:0.05, 1:0.25, in the manufacture of a medicament for the treatment of an ailment, wherein the medicament includes a therapeutic compound and the composition is present in an amount that protects said compound from serine proteases in the intestine.

In an embodiment, the fourth aspect of the invention provides for the use of a composition including EDTA and calcium or magnesium, wherein the molar ratio of calcium or magnesium to EDTA is at least 1:1, and preferably 1:1, 1:2, 1:0.05, 1:0.25, in the manufacture of a medicament for the treatment of an ailment, wherein the medicament includes a therapeutic compound and the composition is present in an amount that protects said compound from serine proteases in the intestine.

In an alternate embodiment, the fourth aspect of the invention provides for the use of a composition including EGTA and calcium or magnesium, wherein the molar ratio of calcium or magnesium to EGTA is at least 1:1, and preferably 1:1, 1:2, 1:0.05, 1:0.25, in the manufacture of a medicament for the treatment of an ailment, wherein the medicament includes a therapeutic compound and the composition is present in an amount that protects said compound from serine proteases in the intestine.

In an alternate preferred embodiment, the invention provides for the use of: (i) a therapeutic compound; and (ii) EDTA or EGTA, or calcium salts thereof, in the manufacture of a medicament, wherein said EDTA or EGTA are present in the composition at a level giving rise to a concentration of 0.15 to 10 mg/ml and the composition is formulated for delivery of the compound in the intestinal tract.

In yet a further alternate preferred embodiment the invention provides for the use of: (i) a therapeutic compound; and (ii) EDTA or EGTA, or calcium salts thereof, in the manufacture of a medicament, wherein said EDTA or EGTA are present in the composition at a level giving rise to a concentration of 0.1 to 10 mg/ml and the composition is formulated for delivery of the compound in the intestinal tract. Preferably, the EDTA or EGTA, or calcium salts thereof, concentration is between 0.15 to 10 mg/ml, more preferably between 0.2 to 5 mg/ml or 0.2 to 1 mg/ml or even more preferably between 0.2 to 0.3 mg/ml. For example, it may be in a concentration of between approximately 0.3 to 10 mg/ml. Alternatively the concentration can be: 0.15, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 or 10.0 mg/ml.

C. Methods for treating a subject

In a fifth aspect, the invention provides a method for treating a subject suffering from an ailment, wherein the subject's treatment is affected by the inhibition of trypsin or other serine protease including chymotrypsin, elastase or DPP4, said method comprising the step of: administering to a subject a composition comprising EDTA and or EGTA alone or in combination with calcium or magnesium, wherein the molar ratio of calcium or magnesium to EDTA or EGTA is at least 1:1. Preferably, the composition is provided in a pharmaceutically or therapeutically effective amount.

In an embodiment, the fifth aspect of the invention provides a method for treating a subject suffering from an ailment, wherein the subject's treatment is affected by the inhibition of trypsin or a serine protease including chymotrypsin, elastase or DPP4, said method comprising the step of: administering to a subject a composition comprising EDTA alone or in combination with calcium or magnesium, wherein the molar ratio of calcium or magnesium to EDTA is at least 1:1, and preferably 1:1, 1:2, 1:0.05, 1:0.25.

In an alternate embodiment, the fifth aspect of the invention provides a method for treating a subject suffering from an ailment, wherein the subject's treatment is affected by the inhibition of trypsin or a serine protease including chymotrypsin, elastase or DPP4, said method comprising the step of: administering to a subject a composition comprising EGTA alone or in combination with calcium or magnesium, wherein the molar ratio of calcium or magnesium to EGTA is at least 1:1, and preferably 1:1, 1:2, 1:0.05, 1:0.25.

As examples of the invention, the above method is used for treatment of a disease of the clotting cascade, for alleviation of pathogenic sequelae of snake bite or spider envenomation, for reducing local effects of inflammation, for combatting the effects of alpha-1 anti-trypsin deficiency (e.g. in the lung), for avoiding localized side-effects of increased trypsin concentrations resulting from pancreatic disease, and for protection from serine proteases of therapeutic proteins introduced into the intestine.

In an alternate preferred embodiment, the invention provides a method for treating a subject suffering from an ailment, wherein the subject's treatment is affected by the inhibition of trypsin or other serine protease including chymotrypsin, elastase and DPP4, said method comprising the step of: administering to a subject EDTA or EGTA, or calcium salts thereof, wherein said EDTA or EGTA are present in the composition at a level giving rise to a EDTA or EGTA concentration of 0.10 to 10 mg/ml. Preferably, the EDTA or EGTA, or calcium salts thereof, concentration is between 0.15 to 10 mg/ml, more preferably between 0.2 to 5 mg/ml or 0.2 to 1 mg/ml or even more preferably between 0.2 to 0.3 mg/ml. For example, it may be in a concentration of between approximately 0.3 to 10 mg/ml. Alternatively the concentration can be: 0.15, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 or 10.0 mg/ml.

In yet a further alternate preferred embodiment, the invention provides a method for treating a subject suffering from an ailment, wherein the subject's treatment is affected by the inhibition of trypsin or other serine protease including chymotrypsin, elastase and DPP4, said method comprising the step of: administering to a subject EDTA or EGTA, or calcium salts thereof, in the manufacture of a medicament, wherein said EDTA or EGTA are present in the composition at a level giving rise to a EDTA or EGTA concentration of 0.3 to 10 mg/ml.

In a sixth aspect, the invention provides a method for treating a subject suffering from an ailment, said treatment comprising administering to a subject a therapeutic compound to treat the ailment and at substantially the same time administering a therapeutically or pharmaceutically acceptable composition comprising EDTA and or EGTA and calcium, wherein the molar ratio of calcium to EDTA or EGTA is at least 1:1, and preferably 1:1, 1:2, 1:0.05, 1:0.25, wherein said composition is present in an amount that protects said compound from at least a serine protease in the intestine.

In an alternate embodiment, the sixth aspect of the invention provides a method for treating a subject suffering from an ailment, said treatment comprising administering to a subject a therapeutic compound to treat the ailment and at substantially the same time administering a therapeutically or pharmaceutically acceptable composition comprising EDTA and calcium, wherein the molar ratio of calcium to EDTA is at least 1:1, and preferably 1:1, 1:2, 1:0.05, 1:0.25, wherein said composition is present in an amount that protects said compound from at least a protease in the intestine.

In an alternate embodiment, the sixth aspect of the invention provides a method for treating a subject suffering from an ailment, said treatment comprising administering to a subject a therapeutic compound to treat the ailment and at substantially the same time administering a therapeutically or pharmaceutically acceptable composition comprising EGTA and calcium, wherein the molar ratio of calcium to EGTA is at least 1:1, and preferably 1:1, 1:2, 1:0.05, 1:0.25, wherein said composition is present in an amount that protects said compound from at least a protease in the intestine.

In an alternate preferred embodiment the invention provides a method for treating a subject suffering from an ailment, said treatment comprising administering to a subject a therapeutic compound to treat the ailment and at substantially the same time administering a therapeutically or pharmaceutically acceptable composition comprising EDTA or EGTA, or calcium salts thereof, wherein said EDTA or EGTA are present in the composition at a level giving rise to a concentration of 0.15 to 10 mg/ml and the composition is formulated for delivery of the compound in the intestinal tract.

In an alternate preferred embodiment the invention provides a method for treating a subject suffering from an ailment, said treatment comprising administering to a subject a therapeutic compound to treat the ailment and at substantially the same time administering a therapeutically or pharmaceutically acceptable composition comprising EDTA or EGTA, or calcium salts thereof, wherein said EDTA or EGTA are present in the composition at a level giving rise to a concentration of 0.3 to 10 mg/ml and the composition is formulated for delivery of the compound in the intestinal tract.

### D. Administration of Pharmaceutical Compositions of the Invention

Pharmaceutical compositions of the invention disclosed herein may be administered either therapeutically or preventively. In a therapeutic application, compositions are administered to a patient already suffering from an ailment, in an amount sufficient to cure or at least partially arrest its symptoms and/or complications. The composition should provide a quantity of the active compound sufficient to effectively treat the patient either in a single dose or as part of a treatment regime.

In a preventative application, compositions of the invention are administered to a subject at risk of developing an ailment, in an amount sufficient to at least partially arrest the ailment's symptoms and/or complications. The composition should provide an amount of active ingredient sufficient to treat the patient.

Preferably, for administration to a subject, the therapeutic or pharmaceutical composition is provided as a pharmaceutically acceptable composition. When in this form, the composition will be pharmaceutical formulated together with one or more pharmaceutically acceptable carriers (additives) and/or diluents.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Methods for preparing administrable compositions are apparent to those skilled in the art, and are described in more detail in, for example, Remington's Pharmaceutical Science, 15th ed., Mack Publishing Company, Easton, Pa., hereby incorporated by reference in its entirety.

As used here, the term “pharmaceutically-acceptable carrier” means a pharmaceutically-acceptable material, composition or vehicle, such as a liquid or solid filler, diluent, excipient, manufacturing aid, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials that can serve as pharmaceutically-acceptable carriers include, but are not limited to: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, binding agents, fillers, lubricants, mucolytic agents, colouring agents, disintegrants, release agents, coating agents, sweetening agents, flavouring agents, perfuming agents, preservative, water, salt solutions, alcohols, antioxidants, polyethylene glycols, gelatin, lactose, amylose, magnesium stearate, talc, silicic acid, viscous paraffin, hydroxymethylcellulose, polyvinylpyrrolidone and the like can also be present in the formulation. The terms such as “excipient”, “carrier”, “pharmaceutically acceptable carrier” or the like are used interchangeably herein.

Examples of pharmaceutically acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

The compositions described herein can additionally contain other adjunct components conventionally found in pharmaceutical compositions, at their art-established usage levels. Thus, for example, the compositions can contain additional, compatible, pharmaceutically-active materials such as, for example, antipruritics, astringents, local anaesthetics or anti-inflammatory agents. However, such materials, when added, should not unduly interfere with the biological activities of the components of the compositions described herein.

As described in detail below, the pharmaceutical acceptable compositions described herein can be specially formulated for administration in solid, gel or liquid form, including those adapted for the following: (1) oral administration, for example, drenches (aqueous or nonaqueous solutions or suspensions), capsules, pills, tablets, granules, pastes for application to the tongue; (2) parenteral administration, for example, by subcutaneous, intramuscular, intravenous or epidural injection as, for example, a sterile solution or suspension, or sustained-release formulation; (3) injection directly into the site needing treatment; (4) topical application, for example, as a cream, lotion, gel, ointment, or a controlled-release patch or spray applied to the skin; (5) intravaginally or intrarectally, for example, as a pessary, cream, suppository or foam; (6) sublingually; (7) ocularly as an eye drop; (8) transdermally; (9) transmucosally; or (10) nasally.

In one embodiment, the pharmaceutical composition of the invention is administered orally, for example, with an inert diluent or an assimilable edible carrier. For oral therapeutic administration, the pharmaceutical composition may be incorporated with excipients and used in the form of ingestible tablets, buccal tablets, troches, capsules, elixirs, suspensions, syrups, wafers, and the like.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition, these oral formulations may contain suitable flavouring and colourings agents.

When used in capsule form the capsules may be coated with compounds such as glyceryl monostearate or glyceryl distearate which delay disintegration. Tablets, troches, pills, capsules and the like can also contain the following: a binder such as gum tragacanth, acacia, corn starch or gelatin; excipients such as dicalcium phosphate; an additional disintegrating agent such as corn starch, potato starch, alginic acid and the like; a lubricant such as magnesium stearate; and a sweetening agent such as sucrose, lactose or saccharin or a flavouring agent such as peppermint, oil of wintergreen, or cherry flavouring.

When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both.

Liquid forms for oral administration (such as a syrup or elixir) can contain, in addition to the above agents, a liquid carrier, a sweetening agent (e.g. sucrose), a preservative (eg methyl and propylparabens), a dye and flavouring such as cherry or orange flavour. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like. The emulsions for oral administration may further comprise one or more emulsifying agents. Suitable emulsifying agents include dispersing agents as exemplified above or natural gums such as guar gum, gum acacia or gum tragacanth.

In addition, the pharmaceutical acceptable composition can be incorporated into sustained-release preparations and formulations. Such pharmaceutical compositions may further include a suitable buffer to minimise acid hydrolysis. Suitable buffer agent agents are well known to those skilled in the art and include, but are not limited to, phosphates, citrates, carbonates and mixtures thereof.

The therapeutically effective amount of a pharmaceutical compositions disclosed herein for any particular subject will depend upon a variety of factors including: the toxicity and therapeutic efficacy of the pharmaceutical composition; the severity of the ailment; the age, body weight, general health, sex and diet of the patient; the time of administration; the route of administration; the rate of sequestration of the compositions; the duration of the treatment; drugs used in combination or coincidental with the treatment, together with other related factors well known in medicine.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices, are preferred.

The data obtained from the cell culture assays described herein can be used in formulating a range of therapeutically effective dosages for use in humans. The dosage of such compounds lies preferably within a range of circulating concentrations that include the ED₅₀ with little or no toxicity. The dosage can vary within this range depending upon the dosage form employed and the route of administration utilized.

In certain embodiments of the invention an effective amount of the composition is given as a single dose of administration. In certain embodiments, the dose is given repeatedly. That is treatment regimens will vary depending on the severity and type of disease, the overall health and age of the patient, and various other conditions to be considered by the treating physician. With respect to duration and frequency of treatment, it is typical for skilled clinicians to monitor subjects to determine when a treatment is providing therapeutic benefit, and to determine whether to increase or decrease dosage, increase or decrease administration frequency, discontinue treatment, resume treatment or make other alteration to treatment regimen.

Pharmaceutical acceptable compositions of the invention described herein, may be provided in a single bolus administration or in multiple doses or treatments and may also be applied by “continuous” therapy where a small amount of the therapeutic composition is provided continually over an extended time period.

Where multiple dosing is used in the treatment (including continuous therapy) the pharmaceutical composition will be administered by a dosing schedule that can vary from once a week to daily depending on several clinical factors, such as the subject's sensitivity to the therapeutic or pharmaceutical composition. The desired dose to be administered in such a regime can be delivered as a single dose at one time or divided into sub-doses, e.g., 2-4 sub-doses and administered over a time period, e.g., at appropriate intervals through the day or other appropriate schedule. Such sub-doses can be administered as unit dosage forms.

In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

The desired dose can be administered using continuous infusion or delivery through a controlled release formulation. In that case, the pharmaceutical composition contained in each sub-dose must be correspondingly smaller to achieve the total daily dosage.

Therapeutic advantages may be realised through combination regimens. In certain embodiments of the invention, the method may further comprise the step of: administering to a subject, at the same time or concomitantly with the treatment identified in the second or third aspects of the invention, a second active agent that is an adjunct treatment for the ailment that the patient is suffering from or may suffer from when delivered preventatively.

The following Examples serve to illustrate the present invention, and should not be construed as limiting.

### EXAMPLES

### Example 1—Inhibition of Trypsin by a Combination of EDTA and Calcium

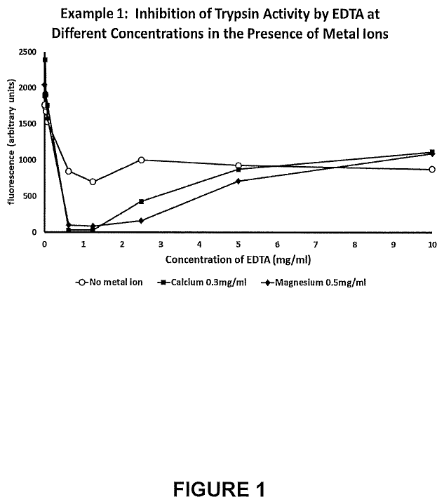

A dilute solution of phosphate-buffered saline (PBS) was prepared at a concentration of 0.3 mM phosphate (pH7), and used to dissolve calcium chloride dihydrate or magnesium chloride at a concentration of 0.3 mg/ml. Trypsin from porcine pancreas was dissolved in the Ca/PBS buffer, in either the presence or absence of calcium, at a concentration of trypsin at 0.02 mg/ml. 5 ml aliquots of disodium EDTA (dihydrate) solution were prepared in PBS with and without calcium or magnesium at doubling dilutions from 8 mg/ml downwards, and the pH of each solution adjusted to 7. Trypsin substrate (namely Z-L-arginine-4-methyl-7-coumarinylamide HCl) was dissolved in DMSO at a concentration of 0.125 mg/ml, then diluted 100-fold in Ca/PBS buffer. EDTA solutions were dispensed into the wells of a black microplate (60 ul per well) then 10 ul of trypsin solution was added, followed by 10 ul of substrate. The reaction was allowed to proceed for 30 minutes, and the extent of reaction determined in a fluorescence microplate reader by measuring the fluorescence of the reaction product (coumarin) at excitation wavelength 365 nm, emission 440 nm. As can be seen in FIG. 1, EDTA has a significant inhibitory effect on the activity of trypsin either alone or in combination with either calcium (0.3 mg/ml) or magnesium (0.5 mg/ml).

### Example 2—Inhibition of Trypsin by EGTA

A dilute solution of phosphate-buffered saline (PBS) was prepared at a concentration of 0.3 mM phosphate (pH7), and used to dissolve calcium chloride dihydrate at a concentration of 0.3 mg/ml. Trypsin from porcine pancreas was dissolved in the Ca/PBS buffer at a concentration of 0.02 mg/ml trypsin. 5 ml aliquots of disodium EGTA (dihydrate) solution were prepared in PBS at doubling dilutions from 8 mg/ml downwards, and the pH of each solution adjusted to 7. Trypsin substrate (namely Z-L-arginine-4-methyl-7-coumarinylamide HCl) was dissolved in DMSO at a concentration of 0.125 mg/ml, then diluted 100-fold in Ca/PBS buffer. EGTA solutions were dispensed into the wells of a black microplate (60 ul per well) then 10 ul of trypsin solution was added, followed by 10 ul of substrate. The reaction was allowed to proceed for 30 minutes, and the extent of reaction determined in a fluorescence microplate reader by measuring the fluorescence of the reaction product (coumarin) at excitation wavelength 365 nm, emission 440 nm.

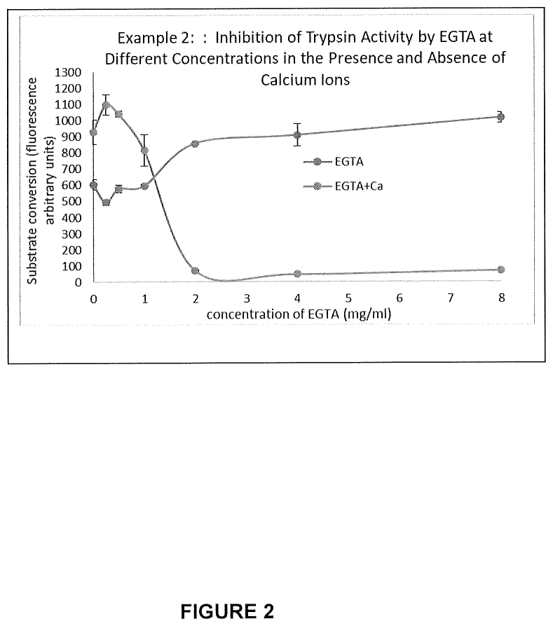

As can be seen from FIG. 2, EGTA has an inhibitory effect on the activity of trypsin.

### Example 3—Effect on Trypsin Activity of a Combination of Phenanthroline and Calcium

A dilute solution of phosphate-buffered saline (PBS) was prepared at a concentration of 0.3 mM phosphate (pH7), and used to dissolve calcium chloride dihydrate at a concentration of 0.3 mg/ml. Trypsin from porcine pancreas was dissolved in the buffer, in either the presence or absence of calcium, at a concentration of 0.02 mg/ml. 5 ml aliquots of disodium EGTA (dihydrate) solution and phenanthroline (Low concentrations of phenanthroline only were employed, because of its limited solubility in aqueous phase) were prepared in PBS with and without calcium at doubling dilutions from 8 mg/ml downwards, and the pH of each solution adjusted to 7. Trypsin substrate (namely Z-L-arginine-4-methyl-7-coumarinylamide HCl) was dissolved in DMSO at a concentration of 0.125 mg/ml, then diluted 100-fold in Ca/PBS buffer. EGTA solutions and phenanthroline were dispensed into the wells of a black microplate (60 ul per well) then 10 ul of trypsin solution was added, followed by 10 ul of substrate. The reaction was allowed to proceed for 30 minutes, and the extent of reaction determined in a fluorescence microplate reader by measuring the fluorescence of the reaction product (coumarin) at excitation wavelength 365 nm, emission 440 nm.

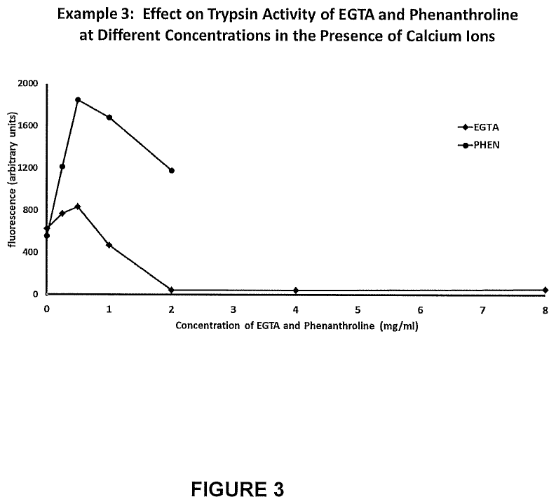

As can be seen from FIG. 3, phenanthroline has no inhibitory effect on trypsin activity. Similar experiments showed that citrate had no effect (results not shown). The fact that neither of these agents has any effect is further proof that the inhibitory actin of EDTA or EGTA is not due simply to chelating activity.

### Example 4—Inhibition of Chymotrypsin by EDTA in the Presence of Calcium Ions

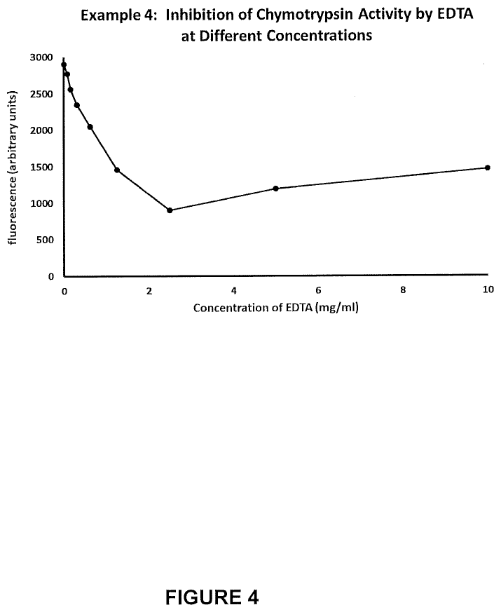

Experiment 1 was repeated, except that chymotrypsin was employed, instead of trypsin, and the experiment was conducted in the presence of calcium ions only. As can be seen from FIG. 4, EDTA has an inhibitory effect on the protease activity of chymotrypsin, in a similar manner to trypsin.

### Example 5—Preparation of a Pharmaceutical Composition Comprising EDTA

A pharmaceutical composition of the present invention comprising an amount of EDTA, or a pharmaceutically acceptable salt thereof (preferably a calcium salt) is prepared for the treatment of a subject in need of treatment. The pharmaceutical composition of the present invention may be used in combination with a therapeutic compound for the treatment of an ailment, disease, disorder or condition in a subject.

In this example, the pharmaceutical composition of the present invention comprising an amount of EDTA, or a pharmaceutically acceptable salt thereof (preferably a calcium salt) is prepared using an amount of EDTA that is capable of producing, in the intestine of a patient, a concentration of EDTA, or a pharmaceutically acceptable salt thereof (preferably a calcium salt) of between 0.1 to 10 mg/ml. Preferably, the concentration of EDTA or a pharmaceutically acceptable salt thereof (preferably a calcium salt) is between 0.15 to 10 mg/ml, more preferably in a concentration of between 0.2 to 5 mg/ml or 0.2 to 1 mg/ml or even more preferably in a concentration of between 0.2 to 0.3 mg/ml.

Thus, for example, in a capsule containing approximately 100 mg of solid powder excipients which dissolve readily in 1 ml or less of liquid situated in the lumen of the gut, there may be provided from 0.2 to 10 mg of EDTA or a pharmaceutically acceptable salt thereof (preferably a calcium salt).

When the composition comprises EDTA and calcium or magnesium, the molar ratio of calcium or magnesium to EDTA is at least 1:1, and preferably 1:1, 1:2, 1:0.05, 1:0.25, wherein the composition is present in an amount that protects a therapeutic compound from at least a protease in the intestine.

The pharmaceutical composition may also comprise a therapeutically effective amount of a general inhibitor of serine protease, such as chenodeoxycholate and/or propyl gallate.

For example, a pharmaceutical composition of the present invention includes a capsule comprising: between 0.2 to 10 mg of EDTA, optionally calcium or magnesium in a molar ratio of calcium to EDTA is at least 1:1, and preferably 1:1, 1:2, 1:0.05, 1:0.25, 70 mg of chenodeoxycholate, and optionally 33 mg of propyl gallate. The capsule may be coated so as to resist breakdown in the stomach, but where the coating dissolves readily at the pH of the duodenum, that is between 5.5 and 6.5.

As discussed above, the pharmaceutical composition of the invention may be used to treatment of an ailment in a subject in need of treatment thereof, wherein the subject is treated with a therapeutic compound.

The therapeutic compound may be selected from any one of insulin; calcitonin; human serum albumin; growth hormone; growth hormone releasing factors; galanin; parathyroid hormone; peptide YY; oxyntomodulin; blood clotting proteins such as kinogen, prothombin, fibrinogen, Factor VII, Factor VIII of Factor IX; erythropoeitins and EPO mimetics; colony stimulating factors including GCSF and GMCSF; platelet-derived growth factors; epidermal growth factors; fibroblast growth factors; transforming growth factors; GLP-1, GLP-2; GLP-1 analogues and fusion proteins, GIP, glucagon; exendin; leptin; GAG; cytokines; insulin-like growth factors; bone- and cartilage-inducing factors; neurotrophic factors; interleukins including IL-I, IL-2, IL-3, IL-4, IL-5, IL-6, IL-7, IL-8, IL-9, IL-10, IL-11, IL-12; interferons including interferon gamma, interferon-1a, interferon alphas; TNF alpha; TNF beta; TGF-beta; cholera toxin A and B fragments; E. coli enterotoxin A and B fragments; secretin; enzymes including histone deacetylase, superoxide dismutase, catalase, adenosine deaminase, thymidine kinase, cytosine deaminase, proteases, lipases, carbohydrases, nucleotidases, polymerases, kinases and phosphatases; transport or binding proteins especially those which bind and/or transport a vitamin, metal ion, amino acid or lipid or lipoprotein such as cholesterol ester transfer protein, phospholipid transfer protein, HDL binding protein; connective tissue proteins such as a collagen, elastin or fibronectin; a muscle protein such as actin, myosin, dystrophin, or mini-dystrophin; a neuronal, liver, cardiac, or adipocyte protein; a cytotoxic protein; a cytochrome; a protein which is able to cause replication, growth or differentiation of cells; a signalling molecule such as an intra-cellular signalling protein or an extracellular signalling protein (eg hormone); trophic factors such as BDNF, CNTF5NGF, IGF, GMF, aFGF, bFGF, VEGF, NT3, T3 and HARP; apolipoproteins; antibody molecules, antibody fragments, single-domain antibodies; receptors in soluble form such as T-cell receptors and receptors for cytokines, interferons or chemokines; proteins or peptides containing antigenic epitopes and fragments; and albumin fusion proteins, derivatives, conjugates and sequence variants of any of the above. These and other proteins may be derived from human, plant, animal, bacterial or fungal sources, and extracted either from natural sources, prepared as recombinants by fermentation or chemically synthesised.

The pharmaceutical composition may be formulated for delivery of a therapeutic compound to the intestinal tract, the pharmaceutical composition comprising (i) the therapeutic compound; and (ii) EDTA, or calcium salts thereof, wherein the EDTA is present in the composition at a level giving rise to an EDTA concentration of 0.15 mg/ml to 10 mg/ml, and chenodeoxycholate and/or propyl gallate. When calcium or magnesium is present, the molar ratio of calcium or magnesium to EDTA is at least 1:1, and preferably 1:1, 1:2, 1:0.05, 1:0.25.

### Example 6—Preparation of a Pharmaceutical Composition Comprising EGTA

A pharmaceutical composition of the present invention comprising an amount of EGTA, or a pharmaceutically acceptable salt thereof (preferably a calcium salt) is prepared for the treatment of a subject in need of treatment. The pharmaceutical composition of the present invention may be used in combination with a therapeutic compound for the treatment of an ailment, disease, disorder or condition.

In this example, the pharmaceutical composition of the present invention comprising an amount of EGTA, or a pharmaceutically acceptable salt thereof (preferably a calcium salt) is prepared using an amount of EGTA that is capable of producing, in the intestine of a patient, a concentration of EGTA, or a pharmaceutically acceptable salt thereof (preferably a calcium salt) of between 0.1 to 10 mg/ml. Preferably, the concentration of EGTA or a pharmaceutically acceptable salt thereof (preferably a calcium salt) is between 0.15 to 10 mg/ml, more preferably in a concentration of between 0.2 to 5 mg/ml or 0.2 to 1 mg/ml or even more preferably in a concentration of between 0.2 to 0.3 mg/ml.

Thus, for example, in a capsule containing approximately 100 mg of solid powder excipients which dissolve readily in 1 ml or less of liquid situated in the lumen of the gut, there may be provided from 0.2 to 10 mg of EGTA or a pharmaceutically acceptable salt thereof (preferably a calcium salt).

When the composition comprises EGTA and calcium or magnesium, the molar ratio of calcium or magnesium to EGTA is at least 1:1, and preferably 1:1, 1:2, 1:0.05, 1:0.25, wherein the composition is present in an amount that protects a therapeutic compound from at least a protease in the intestine.

The pharmaceutical composition may also comprise a therapeutically effective amount of a general inhibitor of serine protease, such as chenodeoxycholate and/or propyl gallate.

For example, a pharmaceutical composition of the present invention includes a capsule comprising: between 0.2 to 10 mg of EGTA, optionally calcium or magnesium in a molar ratio of calcium to EGTA is at least 1:1, and preferably 1:1, 1:2, 1:0.05, 1:0.25, 70 mg of chenodeoxycholate, and optionally 33 mg of propyl gallate. The capsule may be coated so as to resist breakdown in the stomach, but where the coating dissolves readily at the pH of the duodenum, that is between 5.5 and 6.5.

As discussed above, the pharmaceutical composition of the invention may be used to treatment of an ailment in a subject in need of treatment thereof, wherein the subject is treated with a therapeutic compound.

The therapeutic compound may be selected from any one of insulin; calcitonin; human serum albumin; growth hormone; growth hormone releasing factors; galanin; parathyroid hormone; peptide YY; oxyntomodulin; blood clotting proteins such as kinogen, prothombin, fibrinogen, Factor VII, Factor VIII of Factor IX; erythropoeitins and EPO mimetics; colony stimulating factors including GCSF and GMCSF; platelet-derived growth factors; epidermal growth factors; fibroblast growth factors; transforming growth factors; GLP-1, GLP-2; GLP-1 analogues and fusion proteins, GIP, glucagon; exendin; leptin; GAG; cytokines; insulin-like growth factors; bone- and cartilage-inducing factors; neurotrophic factors; interleukins including IL-I, IL-2, IL-3, IL-4, IL-5, IL-6, IL-7, IL-8, IL-9, IL-10, IL-11, IL-12; interferons including interferon gamma, interferon-1a, interferon alphas; TNF alpha; TNF beta; TGF-beta; cholera toxin A and B fragments; E. coli enterotoxin A and B fragments; secretin; enzymes including histone deacetylase, superoxide dismutase, catalase, adenosine deaminase, thymidine kinase, cytosine deaminase, proteases, lipases, carbohydrases, nucleotidases, polymerases, kinases and phosphatases; transport or binding proteins especially those which bind and/or transport a vitamin, metal ion, amino acid or lipid or lipoprotein such as cholesterol ester transfer protein, phospholipid transfer protein, HDL binding protein; connective tissue proteins such as a collagen, elastin or fibronectin; a muscle protein such as actin, myosin, dystrophin, or mini-dystrophin; a neuronal, liver, cardiac, or adipocyte protein; a cytotoxic protein; a cytochrome; a protein which is able to cause replication, growth or differentiation of cells; a signalling molecule such as an intra-cellular signalling protein or an extracellular signalling protein (eg hormone); trophic factors such as BDNF, CNTF5NGF, IGF, GMF, aFGF, bFGF, VEGF, NT3, T3 and HARP; apolipoproteins; antibody molecules, antibody fragments, single-domain antibodies; receptors in soluble form such as T-cell receptors and receptors for cytokines, interferons or chemokines; proteins or peptides containing antigenic epitopes and fragments; and albumin fusion proteins, derivatives, conjugates and sequence variants of any of the above. These and other proteins may be derived from human, plant, animal, bacterial or fungal sources, and extracted either from natural sources, prepared as recombinants by fermentation or chemically synthesised.

The pharmaceutical composition may be formulated for delivery of a therapeutic compound to the intestinal tract, the pharmaceutical composition comprising (i) the therapeutic compound; and (ii) EGTA, or calcium salts thereof, wherein the EGTA is present in the composition at a level giving rise to an EGTA concentration of 0.15 mg/ml to 10 mg/ml, chenodeoxycholate and/or propyl gallate. When calcium or magnesium is present, the molar ratio of calcium or magnesium to EGTA is at least 1:1, and preferably 1:1, 1:2, 1:0.05, 1:0.25.

1. A compound for use as an inhibitor of a serine protease selected from the group comprising trypsin, chymotrypsin, elastase and dipeptidyl peptidase 4 (DPP4), which compound is EDTA or EGTA, or a pharmaceutically acceptable salt thereof.

2. The compound of claim 1 wherein the compound is a calcium or magnesium salt.

3. A product or pharmaceutical composition comprising (a) a compound which inhibits a serine protease selected from the group comprising trypsin, chymotrypsin, elastase, and dipeptidyl peptidase (DPP4), wherein said compound is EDTA or EGTA, or a pharmaceutical salt thereof, chenodeoxycholate and/or propyl gallate; and (b) a therapeutic compound, wherein (a) and (b) are prepared for simultaneous, separate or sequential delivery to a subject for the treatment of a disease or condition affecting the subject, or for preventing a disease or condition affecting the subject.

4. The product or pharmaceutical composition of claim 3, wherein the compound is a calcium or magnesium salt.

5. A product or pharmaceutical composition comprising: (a) a compound according to either of claims 1 and 2, and (b) a therapeutic compound, wherein (a) and (b) are prepared for simultaneous, separate or sequential delivery to a subject for the treatment of a disease or condition affecting the subject, or for preventing a disease or condition affecting the subject.

6. The product according to any one of claims 3-5, wherein the therapeutic compound is selected from the group comprising: insulin; calcitonin; human serum albumin; growth hormone; growth hormone releasing factors; galanin; parathyroid hormone; peptide YY; oxyntomodulin; blood clotting proteins such as kinogen, prothombin, fibrinogen, Factor VII, Factor VIII of Factor IX; erythropoeitins and EPO mimetics; colony stimulating factors including GCSF and GMCSF; platelet-derived growth factors; epidermal growth factors; fibroblast growth factors; transforming growth factors; GLP-1, GLP-2; GLP-1 analogues and fusion proteins, GIP, glucagon; exendin; leptin; GAG; cytokines; insulin-like growth factors; bone- and cartilage-inducing factors; neurotrophic factors; interleukins including IL-I, IL-2, IL-3, IL-4, IL-5, IL-6, IL-7, IL-8, IL-9, IL-10, IL-11, IL-12; interferons including interferon gamma, interferon-1a, interferon alphas; TNF alpha; TNF beta; TGF-beta; cholera toxin A and B fragments; E. coli enterotoxin A and B fragments; secretin; enzymes including histone deacetylase, superoxide dismutase, catalase, adenosine deaminase, thymidine kinase, cytosine deaminase, proteases, lipases, carbohydrases, nucleotidases, polymerases, kinases and phosphatases; transport or binding proteins especially those which bind and/or transport a vitamin, metal ion, amino acid or lipid or lipoprotein such as cholesterol ester transfer protein, phospholipid transfer protein, HDL binding protein; connective tissue proteins such as a collagen, elastin or fibronectin; a muscle protein such as actin, myosin, dystrophin, or mini-dystrophin; a neuronal, liver, cardiac, or adipocyte protein; a cytotoxic protein; a cytochrome; a protein which is able to cause replication, growth or differentiation of cells; a signalling molecule such as an intra-cellular signalling protein or an extracellular signalling protein (eg hormone); trophic factors such as BDNF, CNTF5NGF, IGF, GMF, aFGF, bFGF, VEGF, NT3, T3 and HARP; apolipoproteins; antibody molecules, antibody fragments, single-domain antibodies; receptors in soluble form such as T-cell receptors and receptors for cytokines, interferons or chemokines; proteins or peptides containing antigenic epitopes and fragments; and albumin fusion proteins, derivatives, conjugates and sequence variants of any of the above. These and other proteins may be derived from human, plant, animal, bacterial or fungal sources, and extracted either from natural sources, prepared as recombinants by fermentation or chemically synthesised. In a preferred aspect, the therapeutic compound is a peptide selected from insulin, calcitonin, growth hormone, growth hormone releasing factors, galanin, parathyroid hormone, peptide YY, oxyntomodulin, erythropoeitins, colony stimulating factors, platelet-derived growth factors, epidermal growth factors, fibroblast growth factors, transforming growth factors, GLP-1, GLP-2, GIP, glucagon, exendin, leptin, neurotrophic factors, insulin-like growth factors, cartilage-inducing factors, IL-1, IL-2, IL-3, IL-4, IL-5, IL-6, IL-7, IL-8, IL-9, IL-10, IL-11, IL-12, interferon-gamma, interferon-la, interferon alphas, and conjugates of any of the above, fusion proteins including any of the above, and any of the above in combination.

7. The product or pharmaceutical composition according to claim 5, further comprising chenodeoxycholate and/or propyl gallate.

8. The product or pharmaceutical composition according to any one of claims 3-7, wherein the product is delivered to the gut of the subject.

9. The product or pharmaceutical composition according to claim 8 wherein the compound is present in the intestine in a concentration of between 0.10 to 10 mg/ml.

10. The product or pharmaceutical composition according to claim 8 wherein the compound is present in the intestine in a concentration of between 0.2 to 10 mg/ml.

11. The product or pharmaceutical composition according to claim 8 wherein the compound is present in the intestine in a concentration of between 0.3 to 5 mg/ml.

12. The product or pharmaceutical composition according to claim 8 wherein the compound is present in the intestine in a concentration of between 0.2 to 1 mg/ml.

13. A method of inhibiting a serine protease selected from the group comprising trypsin, chymotrypsin, elastase and DPP4, the method comprising the step of: administering to a subject a compound of any one of claim 1 or 2 or a product or pharmaceutical composition according to any one of claims 3 to 12.

14. Use of: (i) a therapeutic compound; and (ii) EDTA or EGTA, or calcium salts thereof, in the manufacture of a medicament, wherein said EDTA or EGTA are present in the composition at a level giving rise to a concentration of 0.10 to 10 mg/ml and the composition is formulated for delivery of the compound in the intestinal tract.

15. Use of: (i) a therapeutic compound; and (ii) EDTA or EGTA, or calcium salts thereof, in the manufacture of a medicament, wherein said EDTA or EGTA are present in the composition at a level giving rise to a concentration of 0.2 to 10 mg/ml and the composition is formulated for delivery of the compound in the intestinal tract.

16. Use of: (i) a therapeutic compound; and (ii) EDTA or EGTA, or calcium or magnesium salts thereof, in the manufacture of a medicament, wherein said EDTA or EGTA are present in the composition at a level giving rise to a concentration of 0.3 to 5 mg/ml and the composition is formulated for delivery of the compound in the intestinal tract.

17. Use of: (i) a therapeutic compound; and (ii) EDTA or EGTA, or calcium or magnesium salts thereof, in the manufacture of a medicament, wherein said EDTA or EGTA are present in the composition at a level giving rise to a concentration of 0.2 to 1 mg/ml and the composition is formulated for delivery of the compound in the intestinal tract.

18. The use of any one of claims 14-17, wherein the therapeutic compound is selected from the group comprising: insulin; calcitonin; human serum albumin; growth hormone; growth hormone releasing factors; galanin; parathyroid hormone; peptide YY; oxyntomodulin; blood clotting proteins such as kinogen, prothombin, fibrinogen, Factor VII, Factor VIII of Factor IX; erythropoeitins and EPO mimetics; colony stimulating factors including GCSF and GMCSF; platelet-derived growth factors; epidermal growth factors; fibroblast growth factors; transforming growth factors; GLP-1, GLP-2; GLP-1 analogues and fusion proteins, GIP, glucagon; exendin; leptin; GAG; cytokines; insulin-like growth factors; bone- and cartilage-inducing factors; neurotrophic factors; interleukins including IL-I, IL-2, IL-3, IL-4, IL-5, IL-6, IL-7, IL-8, IL-9, IL-10, IL-11, IL-12; interferons including interferon gamma, interferon-1a, interferon alphas; TNF alpha; TNF beta; TGF-beta; cholera toxin A and B fragments; E. coli enterotoxin A and B fragments; secretin; enzymes including histone deacetylase, superoxide dismutase, catalase, adenosine deaminase, thymidine kinase, cytosine deaminase, proteases, lipases, carbohydrases, nucleotidases, polymerases, kinases and phosphatases; transport or binding proteins especially those which bind and/or transport a vitamin, metal ion, amino acid or lipid or lipoprotein such as cholesterol ester transfer protein, phospholipid transfer protein, HDL binding protein; connective tissue proteins such as a collagen, elastin or fibronectin; a muscle protein such as actin, myosin, dystrophin, or mini-dystrophin; a neuronal, liver, cardiac, or adipocyte protein; a cytotoxic protein; a cytochrome; a protein which is able to cause replication, growth or differentiation of cells; a signalling molecule such as an intra-cellular signalling protein or an extracellular signalling protein (eg hormone); trophic factors such as BDNF, CNTF5NGF, IGF, GMF, aFGF, bFGF, VEGF, NT3, T3 and HARP; apolipoproteins; antibody molecules, antibody fragments, single-domain antibodies; receptors in soluble form such as T-cell receptors and receptors for cytokines, interferons or chemokines; proteins or peptides containing antigenic epitopes and fragments; and albumin fusion proteins, derivatives, conjugates and sequence variants of any of the above.

19. The use according to any one of claims 14-18, further comprising chenodeoxycholate and/or propyl gallate.
